(19) United States
(12) Patent Application Publication
(10) Pub. No.: US 2018/0353472 A1
(43) Pub. Date: Dec. 13, 2018

(54) UNIT DOSE FORMULATIONS FOR USE AS AN ANTI-FUGETACTIC AGENT

(71) Applicant: THE GENERAL HOSPITAL CORPORATION d.b.a MASSACHUSETTS GENERAL HOSPITAL, Boston, MA (US)

(72) Inventors: Mark C. Poznansky, Boston, MA (US); Patrick Reeves, Boston, MA (US)

(21) Appl. No.: 15/774,891

(22) PCT Filed: Nov. 9, 2016
(86) PCT No.: PCT/US2016/061205

§ 371 Date: May 9, 2018

Related U.S. Application Data
(60) Provisional application No. 62/253,099, filed on Nov. 9, 2015.

(51) Int. Cl. A61K 31/395; A61K 9/00

(52) U.S. Cl. CPC A61K 31/395; A61K 9/0019

(57) ABSTRACT

Described herein are unit-dose formulations of 1, 1′-[1,4-phenylenebis (methylene)]-bis- 1,4,8,1 l-tetraazacyclotet-radecane (“AMD3100”) and uses thereof as an anti-fugetactic agent.

### BACKGROUND

Cell movement in response to specific stimuli is observed in prokaryotes and eukaryotes. Cell movement seen in these organisms has been classified into three types: chemotaxis or the movement of cells along a gradient towards an increasing concentration of a chemical; negative chemotaxis which has been defined as the movement down a gradient of a chemical stimulus; and chemokinesis or the increased random movement of cells induced by a chemical agent.

Chemotaxis and chemokinesis occur in mammalian cells in response to the class of proteins, called chemokines. Additionally, chemorepellent, or fugetactic, activity has been observed in mammalian cells. For example, some tumor cells secrete concentrations of chemokines that are sufficient to repel immune cells from the site of a tumor (the “fugetactic wall”), thereby reducing the immune system's ability to target and eradicate the tumor. Metastasizing cancer cells may use a similar mechanism to evade the immune system.

### SUMMARY

Anti-fugetactic agents have been described that inhibit the fugetactic activity of tumor cells and allow the patient's immune system to target the tumor (see US 2008/0300165, incorporated herein by reference in its entirety).

1, 1′-[1,4-phenylenebis (methylene)]-bis-1,4,8,11-tetraazacyclotetradecane (“AMD3100,” also known as mozobil/plerixafor) is known to mobilize hematopoetic stem cells and is currently indicated for use in recruitment of hematopoetic cells to the peripheral blood for collection and use in autologous transplantation. The molecular formula of AMD3100 is C₂₈H₅₄N₈, and it has a molecular weight of about 502.9 g/mol. The structure of AMD3100 is provided herein below.

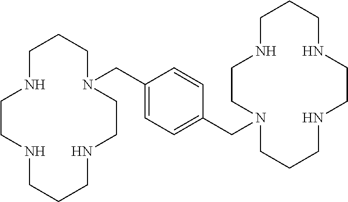 [SMILES: c1cc(CN2CCCNCCNCCCNCC2)ccc1CN1CCCNCCNCCCNCC1]

For the purposes of mobilizing stem cells, AMD3100 is provided as a single use vial containing 1.2 mL of a 20 mg/mL solution for subcutaneous use. In total, the single use vial comprises 24 mg AMD3100 and 5.9 mg sodium chloride in water, adjusted to a pH of 6.0 to 7.5 with hydrochloric acid and sodium hydroxide, as needed. The approved dosage appropriate for recruiting stem cells is 0.24 mg of AMD3100 per kg actual body weight, although pharmacokinetic studies have been conducted on the range of doses selected based on delivering between about 0.04 mg to about 0.24 mg of AMD3100 per kg actual body weight.

Aspects of this disclosure relate to unit-dose formulation of AMD3100 or a pharmaceutically acceptable salt thereof that comprises between about 0.0000001% to about 65% of a dose of AMD3100 that is appropriate for recruiting stem cells. In one embodiment, the unit-dose is between about 0.002 milligrams per kilogram body weight (mg/kg) and about 6 mg/kg per week. In some aspects, this unit-dose formulation may be packaged so as to be administered as a unit-dose. In some embodiments, the unit-dose is administered as a bolus. In some embodiments, the unit-dose is administered by infusion over a period of time.

Such low dose formulations of AMD3100 have an anti-fugetactic effect and may be used in treatment of a tumor or cancer. Repulsion of tumor or cancer antigen-specific T-cells, e.g. from a tumor or cancer expressing high levels of CXCL12 or interleukin 8 (IL-8), allows the tumor or cancer cells to evade immune control. As many as 85% of solid tumors and leukemias express CXCL12 at a level sufficient to have fugetactic effects, e.g. repulsion of immune cells from the tumor. Cancers that express CXCL12 at such levels include, but are not limited to, prostate cancer, lung cancer, breast cancer, pancreatic cancer, ovarian cancer, gastric cancer, esophageal cancer, and leukemia.

### DETAILED DESCRIPTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely examples and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “pharmaceutical formulation” refers to a preparation which is in such form as to permit the active ingredients to be effective, and which contains no additional components which are toxic to the subjects to whom the formulation would be administered.

“Pharmaceutically acceptable” excipients (vehicles, additives) are those which can reasonably be administered to a subject mammal to provide an effective dose of the active ingredient employed.

The terms “patient,” “subject,” “individual,” and the like are used interchangeably herein, and refer to any animal, or cells thereof whether in vitro or in situ, amenable to the methods described herein. In a preferred embodiment, the patient, subject, or individual is a mammal. In some embodiments, the mammal is a mouse, a rat, a guinea pig, a non-human primate, a dog, a cat, or a domesticated animal (e.g. horse, cow, pig, goat, sheep). In especially preferred embodiments, the patient, subject or individual is a human.

The term “treating” or “treatment” covers the treatment of a disease or disorder described herein in a subject, such as a human, and includes: (i) inhibiting a disease or disorder, i.e., arresting its development; (ii) relieving a disease or disorder, i.e., causing regression of the disease or disorder; (iii) slowing progression of the disease or disorder; and/or (iv) inhibiting, relieving, or slowing progression of one or more symptoms of the disease or disorder. For example, treatment of a cancer or tumor includes, but is not limited to, reduction in size of the tumor, elimination of the tumor and/or metastases thereof, remission of the cancer, inhibition of metastasis of the tumor, reduction or elimination of at least one symptom of the cancer, and the like.

The term “administering” or “administration” of an agent, drug, or pharmaceutical composition to a subject includes any route of introducing or delivering to a subject a compound to perform its intended function. Administration can be carried out by any suitable route, including, but not limited to, orally, intranasally, parenterally (intravenously, intramuscularly, intraperitoneally, or subcutaneously), or topically. Administration includes self-administration and the administration by another.

It is also to be appreciated that the various modes of treatment or prevention of medical diseases and conditions as described we intended to mean “substantial,” which includes total but also less than total treatment or prevention, and wherein some biologically or medically relevant result is achieved.

The term “therapeutic” as used herein means a treatment and/or prophylaxis. A therapeutic effect is obtained by suppression, remission, or eradication of a particular disease state. Thus, “sub-therapeutic” when used in relation to dose refers to an amount of an agent that is below amount conventionally deemed to cause the desired, identified therapeutic effect.

As used herein the term “dose appropriate for recruiting stem cells” refers to the doses of AMD3100 indicated, approved, and/or successfully achieving stem cell recruitment, such as, but not limited to, a dose within the range between about 0.04 mg to about 0.24 mg of AMD3100 per kg actual body weight, the commercially available single unit vial of 1.2 mL of 20 mg/mL solution, or the 24 mg of AMD3100 contained therein. Aspects of the disclosure relate to unit-dose formulations of AMD3100 which are sub-therapeutic to such doses appropriate for recruiting stem cells.

By “fugetactic activity” or “fugetactic effect” it is meant the ability of an agent to repel (or chemorepel) a eukaryotic cell with migratory capacity (i.e., a cell that can move away from a repellant stimulus). The term also refers to the chemorepellent effect of a chemokine secreted by a cell, e.g. a tumor cells Usually, the fugetactic effect is present in an area around the cell wherein the concentration of the chemokine is sufficient to provide the fugetactic effect. Some chemokines, including interleukin 8 and CXCL12, may exert fugetactic activity at high concentrations (e.g., over about 100 nM), whereas lower concentrations exhibit no fugetactic effect and may even be chemoattractant.

Accordingly, an agent with fugetactic activity is a “fugetactic agent.” Such activity can be detected using any of a variety of systems well known in the art (see, e.g., U.S. Pat. No. 5,514,555 and U.S. Patent Application Pub. No. 2008/0300165, each of which is incorporated by reference herein in its entirety). A preferred system for use herein is described in U.S. Pat. No. 6,448,054, which is incorporated herein by reference in its entirety.

The term “anti-fugetactic effect” refers to the effect of the anti-fugetactic agent to attenuate or eliminate the fugetactic effect of the chemokine.

“Immune cells” as used herein are cells of hematopoietic origin that are involved in the specific recognition of antigens. Immune cells include antigen presenting cells (APCs), such as dendritic cells or macrophages, B cells, T cells, etc. In one embodiment, immune cells include natural killer cells.

As used herein, the term “unit dose” refers to a single dose of a drug to the patient at the time of administration. In some embodiments, such a unit dose may be packaged in a unit dose container—a non-reusable container designed to hold a quantity of drug intended for administration (other than the parenteral route) as a single dose, directly from the container.

### Formulations and Doses

Aspects of the disclosure relate to a unit-dose formulation of AMD3100 or a pharmaceutically acceptable salt thereof wherein said formulation is packaged so as to be administered as a unit dose.

In one embodiment, the unit-dose formulation can comprise AMD3100 or a pharmaceutically acceptable salt thereof in an amount between about 0.000001% to about 65% of a dose appropriate for recruiting stem cells. Non-limiting examples of unit-dosage formulations are those comprising about 65%, about 55%, about 50%, about 45%, about 40%, about 35%, about 30%, about 25%, about 20%, about 15%, about 10%, about 5%, about 2%, about 1%, about 0.5%, about 0.1%, about 0.05%, about 0.01%, about 0.005%, about 0.001%, about 0.0005%, about 0.0001%, about 0.00005%, about 0.00001%, about 0.000005%, or about 0.000001% of a dose appropriate for recruiting stem cells. Contemplated values include any value, subrange, or range within any of the recited ranges or between any two values, including endpoints.

In some embodiments, the dose appropriate for recruiting stem cells is a dose within the range between about 0.04 mg to about 0.24 mg of AMD3100 or a pharmaceutically acceptable salt thereof per kg actual body weight. For example, for a 62 kg patient, the dose appropriate for recruiting stem cells may be a dose within the range between about 2.48 mg to about 14.88 mg, optionally about 14.88 mg. Thus, the unit-dose formulation for some embodiments herein can be or comprise AMD3100 or a pharmaceutically acceptable salt thereof in an amount between about 0.00000248 mg to about 9.672 mg. Contemplated values include any value, subrange, or range within any of the recited ranges or between any two values, including endpoints.

In some embodiments, the unit-dose formulation can comprise AMD3100 or a pharmaceutically acceptable salt thereof in an amount between about 0.002 mg per kg body weight (mg/kg) and about 6 mg/kg per day. In one embodiment, the unit-dose formulation comprises AMD3100 or a pharmaceutically acceptable salt thereof in an amount between about 0.002 mg/kg and about 4 mg/kg per day, between about 0.002 mg/kg and about 2 mg/kg per day, between about 0.002 mg/kg and about 1 mg/kg per day, between about 0.002 mg/kg and about 0.5 mg/kg per day, between about 0.002 mg/kg and about 0.12 mg/kg per day, between about 0.002 mg/kg and about 0.10 mg/kg per day, between about 0.002 mg/kg and about 0.08 mg/kg per day, between about 0.002 mg/kg and about 0.06 mg/kg per day, between about 0.002 mg/kg and about 0.04 mg/kg per day. In a preferred embodiment, the unit-dose formulation comprises AMD3100 or a pharmaceutically acceptable salt thereof in an amount between about 0.002 mg/kg and about 0.02 mg/kg per day. Contemplated values include any value, subrange, or range within any of the recited ranges, including endpoints.

In general, the unit-dose formulations provided herein can be formulated for administration to a patient by any of the accepted modes of administration. Various formulations and drug delivery systems are available in the art. See, e.g., Remington's Pharmaceutical Sciences, edited by E. W. Martin (Mack Publishing Company, 18th ed., 1990).

In general, unit-dose formulations provided herein will be administered as pharmaceutical compositions by any one of the following routes: topical, enteral, parenteral, intradermal, intramuscular, intraperitoneal, intravenous, subcutaneous, intranasal, buccal, inhalational, epidural or oral application. The nature of the unit-dose formulation will vary based on the route of administration. Thus, in some embodiments, the unit-dose formulation may be, for example, a liquid, a solution, a suspension, an emulsion, an elixir, a syrup, an electuary, a mouthwash, drops, a tablet, a granule, a powder, a lozenge, a pastille, a capsule, a cachet, a pill, an ampoule, a bolus, a suppository, a pessary, a tincture, gel, a paste, an ointment, a cream, a lotion, an oil, a foam, a spray, a mist, a film, an osmotic pump, a bandage, a dressing, a depot, a reservoir, an injection solution or an aerosol.

The unit-dose formulations may be comprised of, in general, AMD3100 or a pharmaceutically acceptable salt thereof, optionally in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Other suitable pharmaceutical excipients and their formulations are described in Remington's Pharmaceutical Sciences, edited by E. W. Martin (Mack Publishing Company, 18th ed., 1990).

The present formulations may, if desired, be presented in a pack or dispenser device containing a unit-dose of the active ingredient. Such a pack or device may, for example, comprise metal or plastic foil, such as a blister pack, or glass, and rubber stoppers such as in vials. The pack or dispenser device may be accompanied by instructions for administration. Compositions comprising a unit-dose formulation of the invention formulated in a compatible pharmaceutical carrier may also be prepared, placed in an appropriate container, and labeled for treatment of an indicated condition.

Other delivery systems can include time-release, delayed release or sustained release delivery systems. Such systems can avoid repeated administrations of the anti-fugetactic agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di- and tri-glycerides; hydrogel release systems; sylastic systems; peptide based systems; wax coatings; compressed tablets using conventional binders and excipients; partially fused implants; and the like.

In one embodiment, the anti-fugetactic agent is administered in a time-release, delayed release or sustained release delivery system. In one embodiment, the time-release, delayed release or sustained release delivery system comprising the anti-fugetactic agent is inserted directly into the tumor. In one embodiment, the time-release, delayed release or sustained release delivery system comprising the anti-fugetactic agent is implanted in the patient proximal to the tumor. Additional implantable formulations are described, for example, in U.S. Patent App. Pub. No. 2008/0300165, which is incorporated herein by reference in its entirety.

In addition, important embodiments of the invention include pump-based hardware delivery systems, some of which are adapted for implantation. Such implantable pumps include controlled-release microchips. A preferred controlled-release microchip is described in Santini, J T Jr. et al., Nature, 1999, 397:335-338, the contents of which are expressly incorporated herein by reference.

### Methods of Treatment

In one aspect of this invention is provided a method for treating cancer in a patient in need thereof by administration of the unit-dose formulation of AMD3100 or a pharmaceutically acceptable salt thereof, which functions as an anti-fugetactic agent.

In one aspect, this invention relates to modulation of a fugetactic effect of a tumor in a patient in need thereof by administration of the unit-dose formulation of AMD3100 or a pharmaceutically acceptable salt thereof. In one embodiment, the tumor expresses an amount of cytokine, e.g. CXCL12, that has a fugetactic effect, preferably on immune cells. Without being bound be theory, it is believed that the anti-fugetactic agents such as the unit-dose formulation of AMD3100 or a pharmaceutically acceptable salt thereof described herein will decrease the fugetactic effect of the tumor such that immune cells can access and attack the tumor or cancer cells.

Cancers or tumors that can be treated by the compounds and methods described herein include, but are not limited to: biliary tract cancer; brain cancer, including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In important embodiments, cancers or tumors escaping immune recognition include glioma, colon carcinoma, colorectal cancer, lymphoid cell-derived leukemia, choriocarcinoma, and melanoma.

In one aspect, an anti-cancer therapy is administered to the patient prior to or following administration of one or more unit doses of AMD3100. Anti-cancer therapies include any such therapies, including but not limited to chemotherapy, radiation therapy, vaccine therapy, immunotherapy, proton beam therapy, and the like. In one embodiment, AMD3100 is administered prior to tumor reduction or ablation surgery (e.g., 6 to 14 hours prior).

In one aspect, the unit-dose of AMD3100 is administered as a single bolus per day. In one embodiment, the unit-dose of AMD3100 is administered once every one, two, three, four, five, six, seven, eight, nine, ten, eleven, or twelve days In one embodiment, the unit dose is administered via two, three, four or more sub-doses per day. In one embodiment, the AMD3100 is administered once a week for 1, 2, 3, 4, 5, 6, 7, 8, 9, or 10 weeks.

In one aspect, the unit-dose is administered via infusion or controlled release. In one embodiment, the unit-dose is administered over 1 to 24 hours. In one embodiment, the patient is administered with AMD3100 for up to 1, 1, 2, 3, or 4 days.

In one aspect, the unit dose of AMD3100 is administered locally to a site of a tumor or near to the tumor. In one embodiment, the unit dose of AMD3100 is administered via a pump, continuous release, or other similar mechanism.

Without being bound by theory, it is contemplated that administration of a unit dose of AMD3100, as described herein, in combination with an immune checkpoint inhibitor will result in a synergy with respect to treatment of a cancer. For example and without limitation, the efficacy of the immune checkpoint inhibitor may be increased when combined with AMD3100 administration.

It is estimated that immune checkpoint inhibitors are effective in about 15% to 20% of patients. In one embodiment, combination treatment with immune checkpoint inhibitors and AMD3100 will improve effectiveness, for example to 30% of patients, 40%, 50%, 60%, 70%, 80%, 90%, 95%, 98%, or 99%, including any range or value between any of these values.

In one embodiment, the effectiveness of the immune checkpoint inhibitor is increased by about 10% to about 500% or more when administered in combination with AMD3100. In one embodiment, the effectiveness is increased by about 10% to about 50%, about 20% to about 60%, about 30% to about 70%, about 40% to about 80%, about 50% to about 90%, about 60% to about 100%, about 80% to about 120%. In one embodiment, the effectiveness is increased by about 20% to about 400%, about 20% to about 300%, about 20% to about 200%, or about 20% to about 100%. Effectiveness may be increased by any value or subrange in any of these ranges, including endpoints.

In one aspect of the invention, the AMD3100 and the immune checkpoint inhibitor are administered sequentially. Preferably, the AMD3100 is administered for a period of time sufficient to have an anti-fugetactic effect, and the immune checkpoint inhibitor is subsequently administered. In one embodiment, the immune checkpoint inhibitor is administered, followed by the AMD3100.

In one aspect of the invention, the immune checkpoint inhibitor is administered after and/or during the period of time of administration of AMD3100. In one embodiment, the immune checkpoint inhibitor is administered during a period of time wherein the fugetactic effect of the cancer cells/tumor is attenuated by AMD3100. The length of time and modes of administration of the immune checkpoint inhibitor will vary, depending on the immune checkpoint inhibitor used, type of tumor being treated, condition of the patient, and the like. Determination of such parameters is within the capability of the skilled clinician.

In one embodiment, administration of AMD3100 and the immune checkpoint inhibitor is alternated. In a preferred embodiment, administration of AMD3100 and the immune checkpoint inhibitor is alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer. In one embodiment, the tumor size does not increase (i.e. progress) for at least a period of time during and/or aftertreatment.

### Immune Checkpoint Inhibitors

As used herein, the term “immune checkpoint inhibitor” refers to agents that promote immune system attack on cancer cells, in particular by blocking proteins made by immune cells and/or cancer cells that prevent the cancer cells from being recognized by the immune system. Where the term immune checkpoint inhibitor is used, it is to be understood that agents targeting similar/associated proteins, e.g. costimulatory molecules, are also included. Anti-cancer therapy that targets immune checkpoints is also known as immune checkpoint therapy. Potential targets for immune checkpoint inhibitors can be found, for example and without limitation, in Topalian, et al., Cancer Cell 27: 450-461 (2015).

In one embodiment, the immune checkpoint inhibitor is an antibody that binds to an immune checkpoint protein or costimulatory protein. In one embodiment, the immune checkpoint inhibitor is a molecule that interferes with binding of immune checkpoint proteins and/or costimulatory proteins and/or co-inhibitory proteins. In one embodiment, the immune checkpoint inhibitor targets an immune checkpoint protein.

In one embodiment, the immune checkpoint protein is programmed death protein-1 (PD-1, also known as CD279 or its ligands programmed death ligand-1 (PD-L1, also known as B7-H1, CD274) and programmed death ligand-2 (PD-L2, also known as B7-DC and CD273). PD-1 is expressed on the surface of activated T cells, B cells, as well as myeloid cells, and inhibits T cells from attacking other cells in the body when bound to PD-L1. PD-L1 and PD-L2 are commonly expressed on the surface of dendritic cells or macrophages. PD-L1 is expressed on many tumors including cancers developing in various organs such as head and neck, lung, stomach, colon, pancreas, breast, kidney, bladder, ovary, cervix, as well as melanoma, glioblastoma, multiple myeloma, lymphoma, and various leukemias. PD-1 inhibitors include, without limitation, pembrolizumab (Keytruda) developed by Merck U.S. and approved for treatment of metastatic melanoma., nivolumab (Opdivo) developed by Bristol-Myers Squibb U.S. and approved in the U.S. for treatment of metastatic melanoma and squamous NSCL cancer, MEDI0680 (AMP-514), and pidilizumab. In some embodiments, the immune checkpoint inhibitor is not pembrolizumab. In some embodiments, the immune checkpoint inhibitor is not nivolumab. PD-L1 inhibitors include, without limitation, BMS-936559, MEDI4736, MSB0010718C, and atezolizumab. Atezolizumab (TECENTRIQ®), developed by Roche, Switzerland (Genentech U.S.) and approved for treatment of the most common type of bladder cancer, i.e., urothelial carcinoma. Atezolizumab is a humanized monoclonal antibody targeting the PD-1 pathway so as to block the immune checkpoint inhibition signaled thereby. The PD-1 pathway refers herein to the signaling of the inhibition of T cell immune responses upon the interaction of the PD-1 and PD-L1/PD-L2. Therapies by the use of other anti-PD-L1 antibodies (e.g., avelumab, durvalumab) for treating various other types of cancers including, for example, non-squamous NSCLC, renal cell carcinoma and bladder cancer, are under investigation and development as well.

In one embodiment, the immune checkpoint protein is cytotoxic T-lymphocyte-associated protein 4 (CTLA4, also known as CD152). CTLA4 is a receptor that is constitutively expressed in Tregs but only upregulated in conventional T cells after activation. CTLA4 binds B7-1 and B7-2 on antigen-presenting cells and serves to inhibit T cell function. CTLA4 inhibitors include, without limitation, ipilibumab, ticilimumab and tremelimumab. In some embodiments, the immune checkpoint inhibitor is not ipilibumab.

In one embodiment, the immune checkpoint protein is B7-1 or B7-2. In one embodiment, the immune checkpoint protein is B7-H3 (also called CD276) or B7x. Molecules that target B7-H3 include, without limitation, enoblituzumab and MGD009.

In one embodiment, the immune checkpoint protein is lymphocyte activation gene 3 (LAG-3, also called CD223). LAG-3 is expressed on activated T cells, NK cells, B cells, and dendritic cells, and binds MHC II. LAG-3 inhibitors include, without limitation, BMS-986016.

In one embodiment, the immune checkpoint protein is a killer inhibitory receptor (KIR). KIRs inhibit NK cell function through interaction with particular MHC I alleles. KIR inhibitors include, without limitation, lirilumab.

In one embodiment, the immune checkpoint protein is T cell ITIM domain (TIGIT). TIGIT inhibitors include, without limitation, MTIG7192A (RG6058).

In one embodiment, the indoleamine 2,3-dioxygenase (IDO) pathway is targeted. Inhibitors of IDO include, without limitation, GDC-0919 and indoximod.

In one embodiment, the immune checkpoint protein is T cell immunoglobulin and mucin-3 (TIM-3), OX40, inducible T cell co-stimulator (ICOS) or its ligand ICOSL, B and T lymphocyte attenuator (BTLA), or V-domain Ig-containing suppressor of T cell activation (VISTA).

In some embodiments, the immune checkpoint activates the protein. For example, activation of OX40, for example with an activating antibody (e.g., MOXR0916, RG7888), may increase T cell differentiation and anti-tumor immunity.

In some embodiments, two or more immune checkpoint inhibitors are administered. In one embodiment, the two more immune checkpoint inhibitors target different proteins and/or pathways.

In some embodiments, AMD3100 and immune checkpoint inhibitor are administered in combination with an additional anti-cancer agent, such as an immunotherapy agent, chemotherapy, radiotherapy, anti-cancer vaccine, etc.

### Kits

Aspects of the disclosure relate to a kit comprising a unit-dose of AMD3100 or a pharmaceutically acceptable salt thereof that comprises between about 0.000001% to about 65% of a dose appropriate for recruiting stem cells and instructions for use as an anti-fugetactic agent. In one embodiment, the unit-dose of AMD3100 or a pharmaceutically acceptable salt thereof comprises between about 0.002 mg/kg and about 6 mg/kg per day, or between about 0.002 mg/kg and about 0.12 mg/kg per day, or any subrange or value within these ranges, including endpoints. Such kits may optionally further comprise a dosing treatment schedule in a readable medium, instructions to a clinician pertinent to the selection of an appropriate dosing regimen based on a particular characteristic of the patient—including, but not limited to age, sex, weight, and cancer or tumor status.

In some embodiments, the kit may include an additional anti-cancer agent. In some embodiments, the additional anti-cancer agent is an immune checkpoint inhibitor.

In some embodiments, the kit may include an accompanying pamphlet or similar written information that accompanies the unit dose form in the kit. In some embodiments, the kit may further comprise electronic, optical, or other data storage, such as a non-volatile memory, for example, to store a digitally-encoded machine-readable representation of such information.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

In some embodiments, the kit includes one or more unit doses, where each unit dose is preloaded into a delivery vehicle. In one embodiment, the delivery vehicle is a sterile syringe. In one embodiment, the delivery vehicle is a nebulizer, inhaler, or similar device. In one embodiment, the delivery vehicle is a cartridge or insert for a syringe, pump, or other delivery device. In one embodiment, the delivery vehicle can be adjusted to deliver the correct unit dose based on body weight of the subject. Such delivery vehicles are known in the art.

### EXAMPLES

### Example 1

Mice are injected with tumor cells (subcutaneous injection) from a tumor that expresses high levels of CXCL12 and a tumor is allowed to develop. Once the tumor has formed, the mice are injected (subcutaneous in the same flank as the tumor) with AMD3100 at a dose between 0.000001% and 65% of the dose appropriate for recruiting stem cells in the selected mouse model or mock control, once a day for 5 days.

One to three days after the final AMD3100 dose, mice are assessed for tumor growth and the reduction or knock down of fugetaxis. It is contemplated that treatment with AMD3100 at a dose sub-therapeutic for recruitment of stem cells will have an anti-fugetactic effect.

### Example 2

A human patient with a breast cancer tumor which over-expresses CXCL12 is administered AMD3100 at a dose between 0.000001% and 65% of the dose appropriate for recruiting stem cells once a day for five days, optionally followed by a chemotherapeutic drug.

It is expected that treatment with AMD3100 will result in increased T cell recruitment into the tumor and/or a decrease in tumor volume and/or an increase in patient survival.

1. A unit-dose formulation of 1′-[1,4-phenylenebis (methylene)]-bis-1,4,8,11-tetraazacyclotetradecane (“AMD3100”) or a pharmaceutically acceptable salt thereof that comprises between about 0.002 mg per kg actual body weight (mg/kg) and about 6 mg/kg, wherein the unit-dose formulation is packaged so as to be administered as a unit-dose.

2. The unit-dose formulation of claim 1, comprising between about 0.002 mg/kg to about 0.12 mg/kg.

3. The unit-dose formulation of claim 1, comprising wherein the unit-dose formulation is suitable for topical, enteral, parenteral, intradermal, intramuscular, intraperitoneal, intravenous, subcutaneous, intranasal, buccal, inhalational, epidural or oral application.

4. The unit-dose formulation of claim 1,
wherein the unit-dose formulation is selected from the group consisting of liquid, solution, suspension, emulsion, elixir, syrup, electuary, mouthwash, drops, tablet, granule, powder, lozenge, pastille, capsule, cachet, pill, ampoule, bolus, suppository, pessary, tincture, gel, paste, ointment, cream, lotion, oil, foam, spray, mist, film, osmotic pump, bandage, dressing, depot, reservoir, injection solution or aerosol.

5. A kit comprising a unit-dose of AMD3100 or a pharmaceutically acceptable salt thereof that comprises between about 0.002 mg per kg actual body weight (mg/kg) and about 6 mg/kg and instructions for use as an anti-fugetactic agent.

6. The kit of claim 5, further comprising an immune checkpoint inhibitor.

7. The kit of claim 5, wherein the kit includes one or more unit doses, where each unit dose is preloaded into a delivery vehicle.
